(19) United States
(12) Patent Application Publication
(10) Pub. No.: US 2007/0276250 A1
(43) Pub. Date: Nov. 29, 2007

(54) Medical device mounting system

(75) Inventor: Brenda L. Donaldson, Harrison Township, MI (US)

Correspondence Address: GE MEDICAL SYSTEM;C/O FOLEY & LARDNER LLP, 777 EAST WISCONSIN AVENUE, MILWAUKEE, WI, 53202-5306, US

(73) Assignee: General Electric Company

(21) Appl. No.: 11/416,960

(22) Filed: May 3, 2006

(51) Int. Cl. A61B 8/14

(52) U.S. Cl. 600/459

(57) ABSTRACT

An imaging apparatus includes an ultrasound sensor array. The imaging apparatus also includes a processor coupled to the ultrasound sensor array, with the processor including device functions. The imaging apparatus also includes a monitor coupled to the processor and producing an image display. The monitor includes a touch screen interface disposed in front of the monitor for operator control over device functions of the ultrasound sensor array and processor. The touch screen interface defines a plurality of activation areas, each activation area associated with at least one device function. The imaging apparatus also includes a mounting system coupled to the monitor and configured to support the monitor in a selected position.

### FIELD OF THE INVENTION

The present invention relates generally to the field of medical imaging apparatuses and specifically to a touch screen imaging apparatus mounting system.

### BACKGROUND OF THE INVENTION

Invasive procedures done in a cardiac catheterization and cardiac electrophysiology lab tend to be complex and involve multiple types of equipment to be attached to or in near proximity to the patient. The care of the patient may involve use of such pieces of equipment as: an x-ray system, a hemodynamic or electrophysiologic monitoring system, a non-invasive blood pressure measurement system, a sterile manifold system, an intra-aortic balloon pump, an intravenous pump, a contrast injection systems, an anesthesia machine, a 3-D electrophysiologic mapping system, a robotic surgical device, and an ultrasound machine. For each case, access to the patient should also be maintained by the physician and circulating nurse or other care provider. Booms may be used to hold monitors which display x-ray, hemodynamic, electrophysiologic, or other data. Other equipment in the room should be able to be moved in order to accommodate patient care. As more pieces of equipment are used, it becomes challenging for the medical staff to have access to both the patient and the equipment. Intravascular ultrasound (IVUS) and intracardiac ultrasound (ICE) are commonly used during some interventional procedures. Physicians who use IVUS, ICE, or other imaging applications such as tissue characterization or strain rate should have access to the patient, the sterile field, and the imaging system. Due to the size, position, and bulk of equipment; typically various pieces of equipment must be moved in and out of the physician's access.

Thus there is a need for a an imaging system that is easily movable into and out of the access of an operator. There is also a need for a movable imaging system with a touch screen in order to reduce the amount of equipment in an area. There is also a need for a method of mounting a touch screen imaging system for easy mobility, access, and viewing.

### SUMMARY OF THE INVENTION

One embodiment of the invention relates to an ultrasound imaging apparatus. The imaging apparatus includes an ultrasound sensor array. The imaging apparatus also includes a processor coupled to the ultrasound sensor array, with the processor including device functions. The imaging apparatus also includes a monitor coupled to the processor and producing an image display. The monitor includes a touch screen interface disposed in front of the monitor for operator control over device functions of the ultrasound sensor array and processor. The touch screen interface defines a plurality of activation areas, each activation area associated with at least one device function. The imaging apparatus also includes a mounting system coupled to the monitor and configured to support the monitor in a selected position.

Another embodiment of the invention relates to a method of mounting a monitor coupled to an ultrasound imaging apparatus. The apparatus includes a processor and the monitor includes a touch screen operator interface. The method of mounting includes the step of coupling the monitor to a mounting system. The mounting system includes a surface mountable base. The mounting system also includes an elongated arm coupled at one end to the base and having a swivel coupled at another end and configured to couple to the monitor. The elongated arm includes a first member and a second member, with one end of each member coupled together in a flexible joint and with at least one member including a telescopic section. The method of mounting also includes the step of coupling the base to a structure.

Another embodiment of the invention relates to a medical imaging apparatus. The medical imaging apparatus includes an image sensor array. The medical imaging apparatus also includes a processor coupled to the sensor array, with the processor including device function sets. The medical imaging apparatus also includes a monitor coupled to the processor and producing an image display. The medical imaging apparatus also includes a touch screen interface disposed in front of the monitor for operator control over device functions of the sensor array and processor. The medical imaging apparatus also includes a plurality of activation areas defined on the touch screen interface, each activation area associated with at least one device function. The medical imaging apparatus also includes a mounting system coupled to the monitor and configured to support the monitor in a selected position.

Another embodiment of the invention relates to a mounting system for a medical device. The medical device includes a monitor having a touch screen operator interface. The mounting system includes a surface mountable base and an elongated arm coupled at one end to the base and having a swivel coupled at another end. The swivel is configured to couple to the monitor. The elongated arm includes a first member and a second member with one end of each member coupled together in a flexible joint. The elongated arm is configured to support the monitor in a selected position.

### BRIEF DESCRIPTION OF THE DRAWINGS

FIG. 1 is a perspective view of a medical imaging apparatus according to one exemplary embodiment.

FIG. 2 is a screen-capture of a display of the medical imaging apparatus of FIG. 1 according to one exemplary embodiment.

### DETAILED DESCRIPTION OF THE PREFERRED EMBODIMENTS

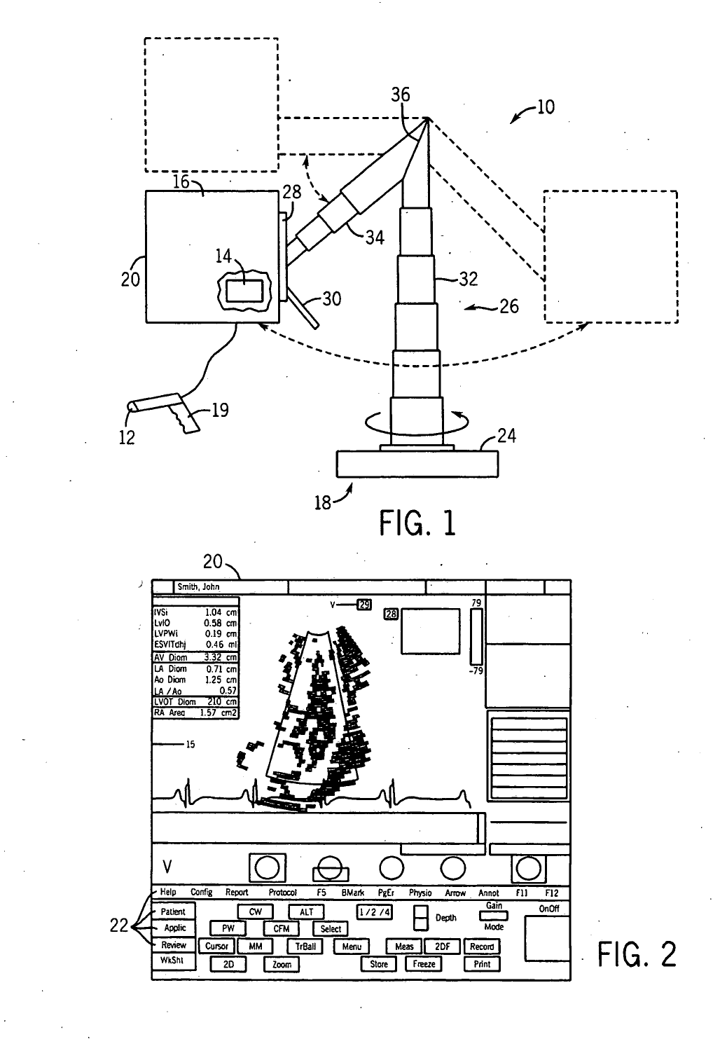

Referring to FIGS. 1 and 2, a medical imaging apparatus 10 is configured to conveniently process or display imaging data retrieved from a subject of interest, for example an anatomical structure. In another exemplary embodiment, medical imaging apparatus 10 may be an ultrasound imaging apparatus that retrieves or displays ultrasound imaging data. In other exemplary embodiments, medical imaging apparatus 10 may be of another imaging technology, for example infrared imaging, or may include another medical device not related to medical imaging, for example a heart monitor.

Medical imaging apparatus 10 generally includes an imaging sensor array 12, a processor 14, a monitor 16, and a mounting system 18. Sensor array 12 is the interface used to transmit or receive data from a subject of interest for use by processor 14. In one exemplary embodiment where imaging apparatus 10 is an ultrasound imaging apparatus, sensor array 12 may be an ultrasound sensor array configured to retrieve ultrasound imaging data from the subject of interest. In other exemplary embodiments, sensor array 12 may be configured to retrieve data related to another imaging technology, for example infrared imaging. In still another exemplary embodiment, sensor array 12 may be coupled to a catheter 19 for retrieval of data from the interior of a subject of interest. In other embodiments, sensor array 12 may be coupled to a device other than catheter 19 for retrieval of data, such as for example, a transthoracic probe and a transophageal probe, which may be used externally or internally relative to a subject of interest in order to retrieve data from it.

Processor 14 is coupled to sensor array 12 and is configured to process or perform operations on data received from sensor array 12. Processor 14 includes a device function set that corresponds to various functions processor 14 may perform on data retrieved from sensor array 12. In various exemplary embodiments the device function set may include any functions related to image processing such as signal transformation, arithmetic, or logic operations. In exemplary embodiments these functions may be used at a higher level for various purposes including to select a portion of a displayed image, zoom in on a portion of a displayed image, isolate a particular signal contributing to a displayed image, or add text to a displayed image. In one exemplary embodiment, processor 14 may be coupled to mounting system 18, for example integrated within monitor 16, while in another exemplary embodiment, processor 14 may be at a location remote from mounting system 18, for example in a computer outside a sterile field around the subject of interest or in another room.

Monitor 16 is coupled to processor 14 and produces an image display based on data received from processor 14. Monitor 16 includes a touch screen interface 20 disposed at the front of monitor 16 for operator control over device functions of imaging sensor array 12 and processor 14. In various exemplary embodiments, monitor 16 may be any past, present, or future monitor that has touch screen capability. Touch screen interface 20 generally defines a plurality of activation areas 22, each of which is associated with at least one device function of the device function set of processor 14 that is activated based on tactile stimulation. In one exemplary embodiment interface 20 may be tactilely stimulated by finger touch. In other exemplary embodiments, interface 20 may be tactilely stimulated by other means such as a cotton swab, plastic pointer, tongue depressant or any other suitable tool. Activation areas 22 may appear as a number of functions including image processing functions and patient specific data.

Mounting system 18 is coupled to monitor 16 and is configured to support monitor 16 in a selected position. Mounting system 18 typically includes a base 24, an elongated arm 26, a swivel 28, and a handle 30. Base 24 is configured to be surface mountable in order to retain mounting system to a surface. In one exemplary embodiment, base 24 may be coupled to a fixed structure, such as a wall, ceiling, column, beam or floor. In another exemplary embodiment, base 24 may be coupled to a movable structure, such as a cart or hospital bed. In still another exemplary embodiment, base 24 may be configured to mount to a surface in a substantially permanent manner, such as by screws, while in other exemplary embodiments base 24 may mount to a surface in a removable or detachable manner, such as by a clamp.

Elongated arm 26 includes a first member 32 and a second member 34. One end of each member 32 and 34 is coupled together in a flexible joint 36. The other end of first member 32 is coupled to base 24 while the other end of second member 34 is coupled to monitor 16 via swivel 28. Second member 34 is configured to move between a position substantially inline with first member 32 and a position substantially parallel to first member 32. First member 32 and/or second member 34 may include a telescopic section that allows for first member 32 and/or second member 34 to extend or retract. In one exemplary embodiment, first member 32 may be configured to rotate about base 24 through 180 degrees, allowing a 180 degree viewing area of monitor 16. In another exemplary embodiment, first member 32 may be configured to rotate about base 24 through 360 degrees, allowing a 360 degree viewing area of monitor 16. In other exemplary embodiments, first member 32 may be fixed to base 24, base 24 may rotate about a surface, or second member 34 may rotate about first member 32.

Swivel 28 couples first member 32 to monitor 16 and is configured to both retain monitor 16 and allow for movement of monitor 16. In one exemplary embodiment, swivel 28 may be a swiveling plate on which monitor 16 rests or is secured that allows for rotation of monitor in a vertical direction. In another exemplary embodiment, swivel 28 may be directly coupled to monitor 16 allowing for movement of monitor 16 in a number of directions on vertical and horizontal axes.

Handle 30 is coupled to swivel 28 and allows for the movement of monitor to a selected position. According to various exemplary embodiments, an operator may apply force to handle 30 and thus move monitor 16 in any suitable direction as allowed by swivel 28, flexible joint 36, and second member 34 depending on the direction and amount of force applied. It is also contemplated that movement can be facilitated with actuators coupled to the members 32, 34 and controlled by wireless transmission, the touch screen 20, or voice activation.

It is noted that each of the individual portions of mounting system l8 may be composed of a number of materials according to various exemplary embodiments including aluminum, steel, other metals, plastic, rubber, glass, or any combination thereof.

Although specific shapes of each element have been set forth in the drawings, each element may be of any other shape that facilitates the function to be performed by that element. For example, many activation areas 22 are shown to have a generally rectangular shape, however, in other exemplary embodiments activation areas 22 may be circular or text-shaped.

Although medical imaging apparatus 10 is illustrated as including multiple features utilized in conjunction with one another, medical imaging apparatus 10 may alternatively utilize less than all of the noted mechanisms or features. For example, in other exemplary embodiments, handle 30 may be omitted with swivel 28 moved via force applied to elongated arm 26.

For purposes of this disclosure, the term “coupled” means the joining of two components (electrical or mechanical) directly or indirectly to one another. Such joining may be stationary in nature or movable in nature. Such joining may be achieved with the two components (electrical or mechanical) and any additional intermediate members being integrally defined as a single unitary body with one another or with the two components or the two components and any additional member being attached to one another. Such joining may be permanent in nature or alternatively may be removable or releasable in nature

The present disclosure has been described with reference to example embodiments, however workers skilled in the art will recognize that changes may be made in form and detail without departing from the spirit and scope of the invention. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example is manifestly intended to be as broad as possible. For example, unless specifically otherwise noted a single particular element may also encompass a plurality of such particular elements.

It is also important to note that the construction and arrangement of the elements of the system as shown in the preferred and other exemplary embodiments is illustrative only. Although only a certain number of embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the assemblies may be reversed or otherwise varied, the length or width of the structures and/or members or connectors or other elements of the system may be varied, the nature or number of adjustment or attachment positions provided between the elements may be varied. It should be noted that the elements and/or assemblies of the system may be constructed from any of a wide variety of materials that provide sufficient strength or durability. Accordingly, all such modifications are intended to be included within the scope of the present disclosure. Other substitutions, modifications, changes and omissions may be made in the design, operating conditions and arrangement of the preferred and other exemplary embodiments without departing from the spirit of the present subject matter.

What is claimed is:

1. An ultrasound imaging apparatus comprising:
an ultrasound sensor array;
a processor coupled to the ultrasound sensor array, with the processor including device functions;
a monitor coupled to the processor and producing an image display, the monitor including a touch screen interface disposed in front of the monitor for operator control over device functions of the ultrasound sensor array and processor, with the touch screen interface defining a plurality of activation areas, each activation area associated with at least one device function; and
a mounting system coupled to the monitor and configured to support the monitor in a selected position.

2. The ultrasound apparatus of claim 1, including a catheter coupled to the sensor array.

3. The ultrasound apparatus of claim 1, wherein the mounting system includes a surface mountable base, an elongated arm coupled at one end to the base and having a swivel coupled at another end, with the swivel configured to couple to the monitor.

4. The ultrasound apparatus of claim 3, wherein the elongated arm includes a first member and a second member, with one end of each member coupled together in a flexible joint.

5. The ultrasound apparatus of claim 4, wherein one of the first and second member includes a telescopic section.

6. The ultrasound apparatus of claim 3, including a handle coupled to the swivel to move the monitor to the selected position.

7. The ultrasound apparatus of claim 3, wherein the surface mountable base is configured to couple to at least one of a fixed structure and a movable structure.

8. The ultrasound apparatus of claim 1, wherein the activation areas include image processing device functions and patient specific data.

9. The ultrasound apparatus of claim 1, wherein the activation areas respond to tactile stimulation other than by finger touch.

10. A method of mounting a monitor coupled to an ultrasound imaging apparatus, with the apparatus including a processor, with the monitor including a touch screen operator interface, the method of mounting comprising:
coupling the monitor to a mounting system, the mounting system including a surface mountable base, an elongated arm coupled at one end to the base and having a swivel coupled at another end, with the swivel configured to couple to the monitor, with the elongated arm including a first member and a second member, with one end of each member coupled together in a flexible joint and with at least one member including a telescopic section; and
coupling the base to a structure.

11. The method of claim 10, including the step of moving the second member from one of a position substantially inline with the first member and a position substantially parallel to the first member.

12. The method of claim 10, including the step of one of extending and retracting the telescopic section.

13. The method of claim 10, wherein the structure is a fixed structure.

14. The method of claim 10, wherein the structure is a movable structure.

15. The method of claim 10, coupling the base to a structure comprises locating the mounting system to a location different and remote from the processor.

16. The method of claim 10, including providing a handle coupled to the swivel to move the monitor to a selected position.

17. A medical imaging apparatus comprising:
an image sensor array;
a processor coupled to the sensor array, with the processor including device function sets;
a monitor coupled to the processor and producing an image display;
a touch screen interface disposed in front of the monitor for operator control over device functions of the sensor array and processor;
a plurality of activation areas defined on the touch screen interface, each activation area associated with at least one device function; and
a mounting system coupled to the monitor and configured to support the monitor in a selected position.

18. The medical apparatus of claim 17, including a catheter coupled to the sensor array.

19. The medical apparatus of claim 17, wherein the mounting system includes a surface mountable base, an elongated arm coupled at one end to the base and having a swivel coupled at another end, with the swivel configured to couple to the monitor.

20. The medical apparatus of claim 19, wherein the elongated arm includes a first member and a second member, with one end of each member coupled together in a flexible joint.

21. The medical apparatus of claim 20, wherein one of the first and second member includes a telescopic section.

22. The medical apparatus of claim 19, including a handle coupled to the swivel to move the monitor to the selected position.

23. The medical apparatus of claim 19, wherein the surface mountable base is configured to couple to one of a fixed structure and a movable structure.

24. The medical apparatus of claim 17, wherein the activation areas include image processing device functions and patient specific data.

25. The medical apparatus of claim 17, wherein the activation areas respond to tactile stimulation other than by finger touch.

26. A mounting system for a medical device, the medical device including a monitor having a touch screen operator interface, the mounting system comprising:
a surface mountable base; and
an elongated arm coupled at one end to the base and having a swivel coupled at another end, with the swivel configured to couple to the monitor, the elongated arm includes a first member and a second member, with one end of each member coupled together in a flexible joint, wherein the elongated arm is configured to support the monitor in a selected position.

27. The mounting system of claim 26, wherein one of the first and second member includes a telescopic section.

28. The mounting system of claim 26, wherein the base is configured to couple to one of a fixed structure and a movable structure.

29. The mounting system of claim 26, wherein the flexible joint is configured to position the first and second member in one of an inline configuration and a substantially parallel configuration.

30. The mounting system of claim 26, including a handle coupled to the swivel and configured to position the monitor.
